(19) United States
(12) Patent Application Publication
(10) Pub. No.: US 2004/0265261 A1
(43) Pub. Date: Dec. 30, 2004

(54) Cleansing emulsion

(75) Inventors: Michaela Kohut, Hamburg (DE); Stephan Ruppert, Hamburg (DE); Jorg Kuther, Pinneberg (DE); Martin Kauffeldt, Hamburg (DE); Olaf Rohde, Wedel (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/830,001

(22) Filed: Apr. 23, 2004

(30) Foreign Application Priority Data
Apr. 24, 2003 (DE) 103 18 526.7

(51) Int. Cl. A61K 7/021; A61K 7/06; A61K 7/11; A61K 7/075

(52) U.S. Cl. 424/70.16; 424/70.24

(57) ABSTRACT

A cosmetic or dermatological cleansing emulsion having a viscosity of from about 500 to about 3,500 mPas and comprising sodium laureth sulfate and/or sodium myreth sulfate, a polyacrylate, a paraffin oil, and an oil having a polarity of about 5 to about 50 mN/m. This abstract is neither intended to define the invention disclosed in this specification nor intended to limit the scope of the invention in any way.

### CROSS-REFERENCE TO RELATED APPLICATIONS

The present application claims priority under 35 U.S.C. § 119 of German Patent Application No. 103 18 526.7, filed on Apr. 24, 2003, the disclosure of which is expressly incorporated by reference herein in its entirety.

### BACKGROUND OF THE INVENTION

1. Field of the Invention

The present invention relates to a cosmetic and/or dermatological cleansing emulsion having a high fat content and small amounts of thickening agents.

2. Discussion of Background Information

The desire for a clean and well-groomed external appearance is probably as old as the human race. Dirty skin and unkempt hair offer an ideal breeding ground and home for pathogens and parasites of all types. The inclination for bodily hygiene has been continuously increased, as in the 20th century in the sixties in addition to “conventional” soap it also became possible to formulate liquid cleansers using newly developed synthetic surfactants. Since then we can no longer imagine our daily life without baths and showers and today a multitude of products are available to the consumer for cleansing the various parts of the body.

Cosmetic and/or dermatological cleansing preparations are as a rule to be applied to the parts of the body to be cleansed in the form of a foam with water. Detergent surfactants are the basis of almost all cosmetic or dermatological cleansing preparations. Surfactants are amphiphilic substances which can dissolve organic, nonpolar substances in water. They are distinguished by ambivalent behavior to water and lipids: the surfactant molecule contains at least one hydrophilic and one lipophilic group each, which make possible the accumulation on the interface between these two classes of substance. In this way, surfactants provide for a reduction in the surface tension of the water, the wetting of the skin, the facilitation of dirt removal and dissolution, easy rinsing off and—if desired—also for foam regulation. The basis for dirt removal of lipid-containing soilings is thus afforded.

The high cleansing power of surfactant-containing cleansing preparations, in particular to lipids, also involves, however, a number of dermatological disadvantages: even in the case of cleansing of the skin with the aid of water—without addition of surfactants—a swelling of the horny layer of the skin initially occurs. The extent of this swelling depends, inter alia, on the duration of the bath and its temperature. At the same time, water-soluble substances are washed off or away, such as, for example, water-soluble dirt constituents, but also substances endogenous to the skin, which are responsible for the water-binding power of the horny layer (“humectants” or “moisturizers”). By means of surface-active substances endogenous to the skin, skin fats are moreover also dissolved to a certain extent and washed out. This causes a subsequent drying out of the skin after initial swelling.

It is understandable that detergent surfactants which are intended to cleanse the skin and hair of fatty and water-soluble dirt constituents also have a defatting action on the normal skin lipids. On each cleansing of the skin, to a varying extent inter-corneocytic lipids and sebum constituents are also removed. This means that the natural water/lipid coat of the skin is more or less disrupted in every washing process. This can lead, particularly in the case of extreme defatting, to a short-term change in the barrier function of the skin, where, of course, the particular condition of the region of the skin treated has a considerable influence on the changes produced. For example, the skin thickness, the number of sebaceous and sweat glands and the sensitivity associated therewith can vary considerably.

In principle, it is accordingly regarded as a requirement of detergent surfactants that they are as biologically inactive as possible in order to avoid undesired side effects. They should display their cleansing action with optimum mildness, best skin compatibility and low defatting.

There has been no lack of attempts to find suitable cleansing preparations which regenerate or “refat” the skin at the same time as having good cleansing power. However, the power achieved often remains below that expected, so that the user as a rule has to resort to separate care products, which are applied to the skin after cleansing and left on it (“leave-on” products).

A number of cleansing preparations, for example shower oils, are known which can be employed for the simultaneous cleansing and refatting of the skin. Refatting cleansing preparations based on emulsions are also known. EP 1166772 A describes, for example, cleansing emulsions having a high oil content.

Preparations of this type have, however, a number of disadvantages:

The foam behavior of the preparations leaves something to be desired. The preparations as a rule foam poorly, i.e. the amount of foam is too low, adversely affecting the cleansing power experienced subjectively by the user and the objective cleansing power of the preparation.

The dispersibility of the preparation on the skin is unsatisfactory. Most viscous, hydrophobic preparations can be dispersed relatively poorly on (water-wetted) skin.

The preparation applied to the skin can be rinsed off again relatively poorly. The rheological properties of the preparations and their polarity are likewise the reason for this.

In order that the emulsion-based cleansing preparations are stable, large amounts of thickeners, for example polyacrylates, must be added to the formulations. Relatively large amounts of polyacrylates, however, worsen the rheological properties of the preparations. The viscosity is increased, whereby the preparations disperse on the skin after application and can be washed off again only with difficulty. The preparations also barely foam. The probable reason for this is that the miscibility with water decreases with increasing content of polyacrylates. In the case of polyacrylate concentrations of below 0.75% by weight of the preparation, however, no stable cleansing emulsions could be formulated hitherto.

The refatting action is defective.

The cleansing preparations simultaneously and relatively unselectively remove the (lipophilic) dirt on the skin as well as the skin's own lipids.

It would be desirable to eliminate or at least alleviate the deficiencies of the known preparations and to have available a cleansing emulsion whose foam quality and amount is increased, and/or whose dispersibility, rinsing-off behavior and/or stability is improved, and/or whose refatting action is strengthened and/or whose selectivity in the removal of lipophilic constituents is increased.

### SUMMARY OF THE INVENTION

The present invention provides a cosmetic and/or dermatological cleansing preparation which comprises (a) from about 2% to about 17% by weight of sodium laureth sulfate and/or sodium myreth sulfate; (b) from about 0.20% to about 0.74% by weight of one or more polyacrylates selected from anionic homopolymers and anionic copolymers of acrylic acid, and/or alkylated acrylic acid and/or esters thereof; and (c) from about 42% to about 51% by weight an oil phase. The oil phase comprises (i) from about 25% to about 50% by weight of one or more paraffin oils, and (ii) from about 0.5% to about 25% by weight one or more oils having a polarity of from about 5 to about 50 mN/m. The emulsion has a viscosity of from about 500 to about 3,500 mPa s at 100 s⁻¹. Unless otherwise stated herein, all percentages are based on the total weight of the emulsion.

In one aspect, the emulsion may comprise from about 4% to about 15% by weight of (a). For example, the emulsion may comprise at least about 5% and/or not more than about 10% by weight of (a).

In another aspect, the emulsion may comprise from about 0.30% to about 0.70% by weight of (b). For example, the emulsion may comprise at least about 0.35% by weight of (b).

In yet another aspect, the emulsion may comprise from about 43% to about 46% by weight of (c). For example, the emulsion may comprise at least about 43% and/or not more than about 45.5% by weight of (c).

In a still further aspect, the emulsion may comprise from about 30% to about 45% by weight of (c)(i) and/or from about 5% to about 20% by weight of (c)(ii).

In a further aspect, the emulsion may comprise at least about 1% by weight of (c)(ii).

In another aspect of the emulsion of the present invention, component (c)(ii) may have a polarity of from about 10 to about 45 mN/m.

In another aspect, component (c)(ii) may comprises at least one fatty acid triglyceride. For example, the fatty acid triglyceride may comprise soybean oil and/or almond oil.

In yet another aspect of the emulsion of the present invention, component (c)(ii) may comprise a hydrocarbon, a fatty acid triglyceride, a silicone oil and/or a carboxylic acid ester.

In a still further aspect, the weight ratio (b): (c)(i) may be from about 1:125 to about 1:68, preferably from about 1:100 to about 1:57.

In yet another aspect, the emulsion of the present invention may have a viscosity of from about 700 to about 3,000 mPa s at 100 s⁻¹.

In a still further aspect, the emulsion of the present invention may further comprise at least one surfactant, preferably a surfactant having an HLB value of higher than about 25, e.g., higher than about 35. In another aspect, the emulsion may comprise from about 1% to about 30% of the surfactant.

In yet another aspect, the emulsion may further comprise at least one active ingredient, for example, in a concentration of from about 0.001% to about 10% by weight.

The present invention also provides a cosmetic and/or dermatological cleansing emulsion which comprises (a) from about 5% to about 10% by weight of sodium laureth sulfate and/or sodium myreth sulfate; (b) from about 0.30% to about 0.70% by weight of one or more polyacrylates selected from anionic homopolymers and anionic copolymers of acrylic acid and/or one or more alkylated acrylic acids and/or an ester thereof, and (c) from about 43% to about 46% by weight an oil phase. The oil phase comprises (i) from about 30% to about 45% by weight of paraffin oil, and (ii) from about 5% to about 20% by weight of one or more oils having a polarity of from about 10 to about 45 mN/m. Furthermore, the emulsion has a viscosity of from about 700 to about 3,000 mPa s at 100 s⁻¹.

In one aspect, the emulsion may comprise at least about 0.35% by weight of (b) and/or not more than about 45.5% by weight of (c).

In another aspect of the emulsion, component (c)(ii) may comprise soybean oil and/or almond oil.

In yet another aspect, the weight ratio (b): (c)(i) may be from about 1:100 to about 1:57.

In a still further aspect, the emulsion may further comprise from about 10% to about 20% by weight of at least one surfactant having an HLB value of higher than about 25 and/or may further comprise from about 0.05% to about 10% by weight of at least one active ingredient.

The present invention also provides various products which comprise the emulsions of the present invention, including the various aspects thereof, e.g., a foamed mousse, a foam bath, a shower bath, a tub bath, a face cleanser, and a hair shampoo. The present invention further provides a compressed gas bottle and an aerosol container which comprise the emulsions.

The present invention also provides a method of cleansing the skin and its appendages (e.g. the hair, nails). The method comprises the application of a product which comprises the emulsion of the present invention onto at least parts of the skin.

Further, the present invention provides a process for making a cosmetic or dermatological cleansing emulsion. This process comprises combining the above components (a) to (c) in the stated amounts to form an emulsion having a viscosity of from about 500 to about 3,500 mPa s at 100 s⁻¹.

According to the present invention, the above components (a) to (c) in the stated amounts and optionally further cosmetic and/or dermatological active ingredients, excipients and additives, can be combined to produce stable cosmetic and/or dermatological cleansing emulsions which have a strongly refatting action on the skin.

Owing to the relatively high oil content, these preparations may moreover have a regenerating action in relation to the general condition of the skin, decrease the feeling of dryness of the skin and make the skin supple.

The viscosity of the emulsions of the present invention may be measured at 25° C. in a DIN cylinder system at a shear rate of 100 l/s.

As a measure of the polarity of the lipophilic components, according to the invention the interfacial tension between a lipid component and water is used. The polarity of the oil phase is the greater, the lower the interfacial tension between this oil phase and water is. According to the invention, the interfacial tension is a possible measure of the polarity of a given oil component.

The interfacial tension is the force which acts on an imaginary line of a length of one meter situated at the interface between two phases. The physical unit for this interfacial tension is conventionally calculated by the relationship force/length and is usually given in mN/m. It has a positive sign if it has the tendency to reduce the interfacial tension. In the converse case, it has a negative sign.

According to the present invention, the polarity may be determined using a KRÜSS digital tensiometer K 10 T, Ring RI 10. It can also be taken and calculated from tables in the manner known to those of skill in the art.

The foam properties of a preparation were determined as follows: The foam measurements were carried out using a modified variant of the Ross-Miles test (DIN 53 902). This modification of the test is described by Dr. F. J. Gohlke (Hoechst AG, Frankfurt) in the journal Parfümerie und Kosmetik No. 3/1964, pages 59-63. In this case, the experimental arrangement is slightly modified, whereby, in comparison with the DIN procedure, somewhat lower foam heights result.

The modification provides for the following experimental design: 50 ml of the surfactant solution to be tested, temperature controlled at 38° C., are introduced into a standing cylindrical vessel. From a defined height, 200 ml of water, likewise temperature controlled at 38° C., with an addition of 300 mg/l of calcium sulfate are allowed to fall onto the surfactant solution. The amount of foam generated thereby is read off immediately, after 30 seconds and after 1, 3, 5, 10 and 15 minutes and plotted in a graph. From the recorded data, the foam stability is additionally calculated and likewise plotted graphically.

In order to determine the refatting properties of the preparation, a washing test was carried out with subsequent lipid analysis. The refatting properties can be determined by the investigation of the lipid residues on the skin.

The stability properties of the preparation may be determined as follows: A formulation is regarded as stable if, after storage at 40° C. for a period of 2 months, no sigificant separation of water and/or oil occurs.

According to the present invention, a cosmetic and/or dermatological cleansing emulsion is preferred which has the following concentration ranges:

a) sodium laureth sulfate and/or sodium myreth sulfate in a total concentration of from about 4% to about 15% by weight,

b) one or more polyacrylates, selected from anionic homo- and/or copolymers of acrylic acid and/or alkylated acrylic acid derivatives and their esters, in a total concentration of from about 0.30% to about 0.70% by weight and particularly preferably of from about 0.35% to about 0.65% by weight,

c) paraffin oil in a total concentration of from about 30% to about 45% by weight and/or

d) one or more oils having a polarity of from about 10 to about 45 mN/m in a total amount of from about 5% to about 20% by weight, in each case based on the total weight of the emulsion.

Likewise, the use of an active ingredient combination of

a) sodium laureth sulfate and/or sodium myreth sulfate in a total concentration of from about 4% to about 15% by weight,

b) one or more polyacrylates, selected from anionic homo- and/or copolymers of acrylic acid and/or alkylated acrylic acid derivatives and their esters, in a total concentration of from about 0.30% to about 0.70% by weight and particularly preferably of from about 0.35% to about 0.65% by weight,

c) paraffin oil in a total concentration of from about 30% to about 45% by weight and/or

d) one or more oils having a polarity of from about 10 to about 45 mN/m in a total amount of from about 5% to about 20% by weight, in each case based on the total weight of the emulsion for the production of stable cosmetic and/or dermatological cleansing emulsions having a strongly refatting action on the skin, is preferred according to the present invention.

A total oil content of from about 42% to about 46% by weight and particularly preferably of from about 43% to about 45.5% by weight, in each case based on the total weight of the preparation, is preferred according to the present invention.

Furthermore, a viscosity of the preparation according to the invention of from about 700 to about 3,000 mPa s at 100 l/s is preferred.

According to the invention, the novel emulsions advantageously have a water content of from about 35% to about 55% by weight, with a water content of from about 40% to about 50% by weight (in each case based on the total weight of the preparation) being preferred.

Advantageous and non-limiting examples of sodium laureth sulfate for use in the present invention are Genapol LRO paste from Clariant, Texapon N 70 from Cognis or Elfan NS 242 Conc. from Akzo Nobel.

Advantageous and non-limiting examples of sodium myreth sulfate for use in the present invention are, for example, Texapon 14S Special from Cognis or Sulfochem ME-60 from Chemron.

Furthermore, a preferred and non-limiting example of a polyacrylate for use in the present invention is Carbopol ETD 2020 from Noveon. This product has the following specifications: viscosity of 47 000-77 000 mPa s at 25° C. and a pH of 5.8-6.3, at 1% concentration in water, Brookfield RVT, 20 rpm.

It is also advantageous if the polyacrylate employed has a viscosity of about 500-1,000 mPa s at a shear rate of 100 l/s at a concentration of 1% and a pH of 5.2-5.6 in a 10% strength aqueous sodium lauryl ether sulfate solution at 25° C.

Acrylate copolymers and/or acrylate/alkyl acrylate copolymers which are obtainable under the trade names Carbopol® 1382, Carbopol® 981 and Carbopol® 5984, Aqua SF-1 from NOVEON Inc. or as Aculyn® 33 from International Specialty Products Corp. are preferred for use in the present invention.

Copolymers of C₁₀₋₃₀ alkyl acrylates and one or more monomers of acrylic acid, of methacrylic acid or of their esters, which are crosslinked with an allyl ether of sucrose or an allyl ether of pentaerithritol are particularly preferred according to the present invention.

Compounds with the INCI designation “Acrylates/C 10-30 alkyl acrylate cross-polymer” are advantageous as well. Those obtainable under the trade names Carbopol ETD 2020, Carbopol 3128, Pemulen TR1 and Pemulen TR2 from NOVEON Inc. are particularly advantageous.

The present emulsions which are characterized in that one or more oils comprised therein have a polarity of about 5 to about 50 mN/m are present, are regarded as advantageous according to the invention.

Constituents of the oil phase of the emulsions of the invention may advantageously be selected from esters of saturated and/or unsaturated, branched and/or linear alkanecarboxylic acids of a chain length of from about 3 to about 30 C atoms and saturated and/or unsaturated, branched and/or linear alcohols of a chain length of from about 3 to about 30 C atoms, from esters of aromatic carboxylic acids and saturated and/or unsaturated, branched and/or linear alcohols of a chain length of from about 3 to about 30 C atoms. Such ester oils can advantageously be selected from, e.g., isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g., jojoba oil.

Furthermore, one or more oil components may advantageously be selected from branched and linear hydrocarbons and hydrocarbon waxes, silicone oils, dialkyl ethers, saturated or unsaturated, branched or linear alcohols, and fatty acid triglycerides, especially the triglycerol esters of saturated and/or unsaturated, branched and/or linear alkanecarboxylic acids of a chain length of from about 8 to about 24, in particular from about 12 to about 18 C atoms. The fatty acid triglycerides can, for example, advantageously be selected from synthetic, semisynthetic and natural oils such as, e.g., olive oil, sunflower oil, soybean oil, groundnut oil, rapeseed oil, almond oil, palm oil, coconut oil, palm kernel oil and the like.

Any desired mixtures of such oil and wax components may also advantageously be employed within the scope of the present invention. It may also be advantageous to employ waxes, for example cetyl palmitate, as the sole lipid component of the oil phase.

According to the invention, the oil component may furthermore advantageously be selected from 2-ethylhexyl isostearate, octyldodecanol, isotridecyl iso-nonanoate, isoeicosane, 2-ethylhexyl cocoate, C₁₂₋₁₅-alkyl benzoate, caprylic/capric acid triglyceride, and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are also advantageous according to the invention.

Fatty acid triglycerides, in particular soybean oil and/or almond oil, may particularly preferably be employed according to the invention as oils having a polarity of from about 5 to about 50 mN/m.

Of the hydrocarbons, paraffin oil, squalane and squalene may advantageously be used within the scope of the present invention.

Advantageously, the oil component may further comprise cyclic or linear silicone oils, in which case, however, it is preferred to use an additional content of other oil-phase components apart from the silicone oil or silicone oils.

Advantageously, cyclomethicone (octamethylcyclotetrasiloxane) is employed as the silicone oil for use in the present invention. However, other silicone oils may also advantageously be used within the scope of the present invention, for example, hexamethylcyclotrisiloxane, polydimethylsiloxane, and poly(methylphenyl-siloxane). Mixtures of cyclomethicone and isotridecyl isononanoate, of cyclomethicone and 2-ethylhexyl isostearate are particularly advantageous.

The oil component may furthermore advantageously be selected from the phospholipids. The phospholipids are phosphoric acid esters of acylated glycerols. Of very great importance among the phosphatidylcholines are, for example, the lecithins, which are represented by the formula

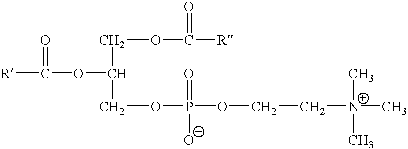

where R′ and R″ are typically linear aliphatic radicals having 15 or 17 carbon atoms and up to 4 cis double bonds.

As a paraffin oil which is advantageous according to the invention, it is possible to employ, for example, Merkur Weissoel Pharma 40 from Merkur Vaseline, Shell Ondina 917, Shell Ondina 927, Shell Oil 4222, Shell Ondina 933 from Shell & DEA Oil, Pionier 6301 S, Pionier 2071 from Hansen & Rosenthal.

A total content of lipophilic components (paraffin oil and polar oils) of from about 43% to about 50% by weight, based on the total weight of the preparation, is advantageous according to the present invention.

The weight ratio of the total amounts of polyacrylates and paraffin oil in the emulsions of the present invention may advantageously be from about 1:125 to about 1:68 and preferably from about 1:100 to about 1:57.

According to the invention, apart from the above-mentioned substances the emulsions of the present invention may optionally contain one or more additives which are customary in cosmetics or dermatology, for example, perfume, colorants, antimicrobial substances, refatting agents, complexing and sequestering agents, pearl luster agents, plant extracts, vitamins, active ingredients, preservatives, bactericides, coloring pigments, thickening agents, plasticizing, moisturizing and/or humectant substances, or other customary constituents of a cosmetic or dermatological formulation such as, e.g., alcohols, polyols, polymers, foam stabilizers, electrolytes, organic solvents and/or silicone derivatives.

Moreover, the preparations according to the invention may contain further surfactants. It is advantageous according to the invention if, as surfactants, anionic, cationic, nonionic and/or amphoteric surfactants are employed. Ionic surfactants, i.e., anionic, cationic and/or amphoteric surfactants, may preferably be employed according to the invention.

Advantageous detergent anionic surfactants within the meaning of the present invention include acylamino acids and their salts such as, e.g.,

acylglutamates, in particular sodium acylglutamate,

sarcosinates, for example myristoyl sarcosine, TEA-lauroyl sarcosinate, sodium lauroyl sarcosinate and sodium cocoyl sarcosinate,

sulfonic acids and their salts such as, e.g.,

acylisethionates, e.g. sodium/ammonium cocoyl isethionate,

sulfosuccinates, for example dioctylsodium sulfosuccinate, disodium laureth sulfosuccinate, disodium lauryl sulfosuccinate and disodium undecyleneamido MEA-sulfosuccinate, disodium PEG-5 lauryl citrate sulfosuccinate and derivatives thereof,

sulfuric acid esters such as, e.g.,

alkyl ether sulfates, for example sodium, ammonium, magnesium, MIPA, TIPA laureth sulfate, sodium myreth sulfate and sodium C₁₂₋₁₃ pareth sulfate,

alkyl sulfates, for example sodium, ammonium and TEA lauryl sulfate.

Further examples of advantageous anionic surfactants include

taurates, for example sodium lauroyl taurate and sodium methyl cocoyl taurate,

ether carboxylic acids, for example sodium laureth-13 carboxylate and sodium PEG-6 cocamide carboxylate, sodium PEG-7-olive oil carboxylate

phosphoric acid esters and salts, such as, for example, DEA-oleth-10 phosphate and dilaureth-4 phosphate,

alkyl sulfonates, for example sodium coconut monoglyceride sulfate, sodium C₁₂₋₁₄ olefin sulfonate, sodium lauryl sulfoacetate and magnesium PEG-3 cocoamide sulfate,

acylglutamates such as di-TEA-palmitoyl aspartate and sodium caprylic/capric glutamate,

acylpeptides, for example palmitoyl hydrolyzed milk protein, sodium cocoyl hydrolyzed soybean protein and sodium/potassium cocoyl hydrolyzed collagen,

and carboxylic acids and derivatives thereof such as, e.g.,

lauric acid, aluminum stearate, magnesium alkanolate and zinc undecylenate,

ester carboxylic acids, for example calcium stearoyl lactylate, laureth-6 citrate and sodium PEG-4 lauramide carboxylate,

alkylaryl sulfonates.

Advantageous detergent cationic surfactants for use in the present invention include quaternary surfactants. Quaternary surfactants contain at least one N atom, which is covalently bonded to 4 alkyl or aryl groups. Alkylbetaine, alkylamido-propylbetaine and alkylamidopropylhydroxysultaine, for example, are advantageous.

Other advantageous cationic surfactants for use in the present invention are furthermore

alkylamines,

alkylimidazoles and

ethoxylated amines

and in particular, their salts.

Advantageous detergent amphoteric surfactants for use in the present invention include acyl/dialkylethylenediamines, for example sodium acylamphoacetate, disodium acylamphodipropionate, disodium alkylamphodiacetate, sodium acyl-amphohydroxypropylsulfonate, disodium acylamphodiacetate, sodium acylampho-propionate and N-coconut fatty acid amidoethyl-N-hydroxyethylglycinate sodium salts.

Further advantageous amphoteric surfactants include N-alkylamino acids, for example aminopropylalkylglutamide, alkylaminopropionic acid, sodium alkylimidodipropionate and lauroamphocarboxyglycinate.

Advantageous detergent nonionic surfactants for use in the present invention include

alkanolamides, such as cocamides MEA/DEA/MIPA,

esters which are formed by esterification of carboxylic acids with ethylene oxide, glycerol, sorbitan or other alcohols,

ethers, for example ethoxylated alcohols, ethoxylated lanolin, ethoxylated polysiloxanes, propoxylated POE ethers and alkyl polyglycosides such as lauryl glucoside, decyl glycoside and coconut glycoside.

Further advantageous nonionic surfactants include alcohols and amine oxides, such as cocoamidopropylamine oxide.

It is advantageous to select the detergent surfactant(s) from surfactants which have an HLB of more than about 25; those which have an HLB of more than about 35 are particularly advantageous.

It is advantageous according to the invention if one or more of these surfactants are employed in a concentration of from about 1% to about 30% by weight, preferably in a concentration of from about 5% to about 25% by weight, and very particularly preferably in a concentration of from about 10% to about 20% by weight, in each case based on the total weight of the preparation.

Furthermore, polysorbates may advantageously be incorporated according to the invention into the preparation as detergent agents.

Advantageous polysorbates include

polyoxyethylene (20) sorbitan monolaurate (Tween 20, CAS No. 9005-64-5)

polyoxyethylene (4) sorbitan monolaurate (Tween 21, CAS No. 9005-64-5)

polyoxyethylene (4) sorbitan monostearate (Tween 61, CAS No. 9005-67-8)

polyoxyethylene (20) sorbitan tristearate (Tween 65, CAS No. 9005-71-4)

polyoxyethylene (20) sorbitan monooleate (Tween 80, CAS No. 9005-65-6)

polyoxyethylene (5) sorbitan monooleate (Tween 81, CAS No. 9005-65-5)

polyoxyethylene (20) sorbitan trioleate (Tween 85, CAS No. 9005-70-3).

In particular,

polyoxyethylene (20) sorbitan monopalmitate (Tween 40, CAS No. 9005-66-7)

polyoxyethylene (20) sorbitan monostearate (Tween 60, CAS No. 9005-67-8)

are particularly advantageous.

If used, the polysorbates may advantageously be employed, individually or as a mixture of two or more polysorbates, in a concentration of from about 0.1% to about 5% by weight and in particular, in a concentration of from about 1.5% to about 2.5% by weight, based on the total weight of the formulation.

The addition of conditioning agents may prove advantageous as well. Suitable examples of the conditioning agents include polymeric quaternary ammonium compounds; cationic cellulose derivatives and polysaccharides. Furthermore, silicone compounds may also be employed for conditioning. Advantageous conditioning agents for use in the invention may, for example, be selected from the compounds listed in the following table.

TABLE 1

| Name according to | | | Example |
|---|---|---|---|
| INCI | CAS number | Polymer type | (trade name) |
| Polyquaternium-2 | CAS 63451-27-4 | Urea, N,N′-bis[3-(dimethylamino) | Mirapol ® A-15 |
| | | propyl]-, | |
| | | polymer with 1,1′-oxybis- | |
| | | (2-chloroethane) | |
| Polyquaternium-5 | CAS 26006-22-4 | Acrylamide, β-methacryloxyethyltriethyl- | |
| | | ammonium methosulfate | |
| Polyquaternium-6 | CAS 26062-79-3 | N,N-dimethyl-N-2- | Merquat ® 100 |
| | | propenyl-2-propenaminium | |
| | | chloride | |
| Polyquaternium-7 | CAS 26590-05-6 | N,N-dimethyl-N-2- | Merquat ® S |
| | | propenyl-2-propenaminium | |
| | | chloride, | |
| | | 2-propenamide | |
| Polyquaternium-10 | CAS 53568-66-4, | Quaternary ammonium salt | Celquat ® SC- |
| | 55353-19-0, 54351- | of hydroxyethyl-cellulose | 230M, polymer |
| | 50-7, 68610-92-4, | | JR 400 |
| | 81859-24-7 | | |
| Polyquaternium-11 | CAS 53633-54-8 | Vinylpyrrolidone/dimethylaminoethyl | Gafquat ® 755N |
| | | methacrylate | |
| | | copolymer/diethyl sulfate | |
| | | reaction product | |
| Polyquaternium- 16 | CAS 29297-55-0 | Vinylpyrrolidone/vinyl- | Luviquat ® HM |
| | | imidazolinium methochloride | 552 |
| | | copolymer | |
| Polyquaternium-17 | CAS 90624-75-2 | | Mirapol ® AD-1 |
| Polyquaternium-19 | CAS 110736-85-1 | Quaternized water-soluble | |
| | | polyvinyl alcohol | |
| Polyquaternium-20 | CAS 110736-86-2 | Water dispersible | |
| | | quaternized polyvinyl | |
| | | octadecyl ether | |
| Polyquaternium-21 | | Polysiloxanepoly- | Abil ® B 9905 |
| | | dimethyldimethyl- | |
| | | ammonium acetate | |
| | | copolymer | |
| Polyquaternium-22 | CAS 53694-17-0 | Dimethyldiallyl- | Merquat ® 280 |
| | | ammonium | |
| | | chloride/acrylic acid | |
| | | copolymer | |
| Polyquaternium-24 | CAS 107987-23-5 | Polymeric quaternary | Quartisoft ® |
| | | ammonium salt of | LM-200 |
| | | hydroxyethylcellulose | |
| Polyquaternium-28 | CAS 131954-48-8 | Vinylpyrrolidone/methacrylamidopropyl- | Gafquat ® HS- |
| | | trimethylammonium | 100 |
| | | chloride copolymer | |
| Polyquaternium-29 | CAS 92091-36-6, | Chitosan which has been | Lexquat ® CH |
| | 148880-32 | reacted with propylene | |
| | | oxide and quaternized with | |
| | | epichlorohydrin | |
| Polyquaternium-31 | CAS 136505-02-7, | Polymeric, quaternary | Hypan ® QT |
| | 139767-67-7 | ammonium salt which is | 100 |
| | | prepared by the reaction of | |
| | | DMAPA acrylate/acrylic | |
| | | acid/acrylo-nitrogen | |
| | | copolymers and diethyl | |
| | | sulfate | |
| Polyquaternium-32 | CAS 35429-19-7 | N,N,N-trimethyl-2-[(2- | |
| | | ethyl-1-oxo-2-propenyl)- | |
| | | oxy]ethanaminium | |
| | | chloride, polymer with | |
| | | 2-propenamide | |
| Polyquaternium-37 | CAS 26161-33-1 | | |
| Polyquaternium-44 | | Copolymeric quaternary | |
| | | ammonium salt of | |
| | | vinylpyrrolidone and | |
| | | quaternized imidazoline | |

Further non-limiting examples of advantageous conditioning agents for use according to the invention include cellulose derivatives and quaternized guar gum derivatives, in particular guar hydroxypropylammonium chloride (e.g. Jaguar Excel®, Jaguar C 162® from Rhodia, CAS 65497-29-2, CAS 39421-75-5).

Nonionic poly-N-vinylpyrrolidone/polyvinyl acetate copolymers (e.g. Luviskol VA 64W®, BASF), anionic acrylate copolymers (Luviflex soft®, BASF), and/or amphoteric amide/acrylate/methacrylate copolymers (e.g. Amphomer®, National Starch) can also be employed advantageously as conditioners.

Generally, an addition of powder raw materials may also be advantageous. The use of talc is particularly preferred.

Ethoxylated glycerol fatty acid esters may be employed for various purposes in aqueous cleansing recipes. Low-ethoxylated glycerol fatty acid esters (EO 3-12) are customarily used as refatting agents for improving the skin sensation after drying off, glycerol fatty acid esters having a degree of ethoxylation of about 30-50 are used as solubilizers for nonpolar substances such as perfume oils. Highly ethoxylated glycerol fatty acid esters are employed as thickeners. It is common to all these substances that they produce a particular skin sensation on the skin on application, i.e., on dilution with water. It has been found that these substances neutralize both the unacceptable sensory functions on washing (caused by the gel-forming agents), and, after drying off, cause a pleasant skin sensation for the consumer. They counteract not only the drying out due to the high amounts of surfactant, but also improve the sensory functions noticeably.

An additional content of antioxidants is in general preferred. According to the invention, non-limiting examples of antioxidants which can be used are all antioxidants which are suitable or customary for cosmetic and/or dermatological applications.

Advantageously, the antioxidants may be selected from amino acids (e.g. glycine, histidine, tyrosine, tryptophan) and their derivatives, imidazoles (e.g. urocaninic acid) and their derivatives, peptides such as D,L-carnosine, D-carnosine, L-carnosine and their derivatives (e.g. anserine), carotenoids, carotenes (e.g. α-carotene, β-carotene, ψ-lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters), and their salts, dilauryl thiodipropionate, distearyl thiodipropionate, thiodipropionic acid and its derivatives (esters, ethers, peptides, lipids, nucleotides, nucleosides and salts), and sulfoximine compounds (e.g. buthionine sulfoximine, homocysteine sulfoximine, buthionine sulfone, penta-, hexa-, heptathionine sulfoximine) in very low tolerable doses (e.g. pmol to μmol/kg), furthermore (metal) chelators (e.g., α-hydroxy fatty acids, palmitic acid, phytic acid, lactoferrin), α-hydroxy acids (e.g., citric acid, lactic acid, malic acid), humic acid, bile acid, bile extracts, bilirubin, biliverdin, EDTA, EGTA and their derivatives, unsaturated fatty acids and their derivatives (e.g., γ-linolenic acid, linoleic acid, oleic acid), folic acid and its derivatives, furfurylidenesorbitol and its derivatives, ubiquinone and ubiquinol and their derivatives, vitamin C and derivatives (e.g., ascorbyl palmitate, Mg ascorbyl phosphate, ascorbyl acetate), tocopherols and derivatives (e.g., vitamin E acetate), vitamin A and derivatives (e.g., vitamin A palmitate), and coniferyl benzoate of benzoin resin, rutic acid and its derivatives, α-glycosylrutin, ferulic acid, furfurylideneglucitol, carnosine, butylhydroxytoluene, butylhydroxy-anisole, nordihydroguaiaretic acid, trihydroxy-butyrophenone, uric acid and its derivatives, mannose and its derivatives, zinc and its derivatives (e.g., ZnO, ZnSO₄), selenium and its derivatives (e.g., selenomethionine), stilbenes and their derivatives (e.g., stilbene oxide, trans-stilbene oxide) and the derivatives suitable according to the invention (salts, esters, ethers, sugars, nucleotides, nucleosides, peptides and lipids) of these active ingredients.

The amount of the above-mentioned antioxidants (one or more compounds) in the emulsions is preferably from about 0.001% to about 30% by weight, particularly preferably from about 0.05% to about 20% by weight, in particular from about 0.1% to about 10% by weight, based on the total weight of the preparation.

If the antioxidant(s) comprise(s) vitamin E and/or its derivatives, their respective concentrations may advantageously be from about 0.001% to about 10% by weight, based on the total weight of the formulation.

If the antioxidant(s) comprise vitamin A and/or vitamin A derivatives, and/or carotenes and/or their derivatives, their respective concentrations may advantageously be from about 0.001% to about 10% by weight, based on the total weight of the formulation.

It has been found that all sorts of active ingredients having differing solubility can be incorporated homogeneously into the cleansing emulsions of the present invention. The substantivity of the active compounds on skin and hair is significantly higher from the present cleansing emulsions than from conventional surfactant-containing cleansing formulations. Without wishing to be bound by any theory, it is assumed that the washing out of the active compounds from the skin by the surfactants contained in the formula is prevented or at least reduced by the formation of an oil film on the skin, such that a larger amount of the active ingredients contained in the product remains on the skin.

According to the invention, the active ingredients (one or more compounds) are very advantageously selected from the following group: acetylsalicylic acid, atropine, azulene, hydrocortisone and its derivatives, e.g., hydrocortisone 17-valerate, vitamins of the B and D series, preferably vitamin B₁, vitamin B₁₂, vitamin D₁, vitamin A and its derivatives such as retinyl palmitate, vitamin E and its derivatives such as, e.g., tocopheryl acetate, vitamin C and its derivatives such as, for example, ascorbyl glucoside, niacinamide, panthenol, bisabolol, polydocanol, unsaturated fatty acids, especially the essential fatty acids (often also called vitamin F), in particular γ-linolenic acid, oleic acid, eicosapentaenoic acid, docosahexaenoic acid and their derivatives, chloramphenicol, caffeine, prostaglandins, thymol, camphor, squalene, extracts and other products of vegetable and animal origin, e.g., evening primrose oil, borage oil or currant pip oil, fish oils, cod liver oil, ceramides and ceramide-like compounds, frankincense extract, green tea extract, water lily extract, liquorice extract, Hamamelis, antidandruff active ingredients (e.g., selenium disulfide, zinc pyrithione, piroctone, olamine, climbazole, octopirox, polydocanol and their combinations), complex active ingredients such as, for example, those from γ-oryzanol and calcium salts such as calcium pantothenate, calcium chloride, and calcium acetate.

It may also be advantageous to select the active ingredients from refatting substances, for example, purcellin oil, Eucerit® and Neocerit®.

Particularly advantageously, the active ingredient(s) may further be selected from NO synthase inhibitors, in particular if the preparations according to the invention are intended to be used for the treatment and prophylaxis of the symptoms of intrinsic and/or extrinsic skin ageing, and for the treatment and prophylaxis of the harmful effects of ultraviolet radiation on the skin. A preferred NO synthase inhibitor is nitroarginine.

Additionally advantageously, the active ingredient(s) may be selected from catechols and bile esters of catechols and aqueous and organic extracts of plants and parts of plants which contain catechols or bile acid esters of catechols, such as, for example, the leaves of the plant family Theaceae, in particular of the species Camellia sinensis (green tea). Their typical ingredients (such as, for example, polyphenols or catechols, caffeine, vitamins, sugars, minerals, amino acids, lipids) are particularly advantageous.

Catechols are a group of compounds which may be considered to be hydrogenated flavones or anthocyanidins and derivatives of “catechol” (3,3′,4′,5,7-flavanpentaol, 2-(3,4-dihydroxyphenyl)chroman-3,5,7-triol). Epicatechol ((2R,3R)-3,3′,4′,5,7-flavan-pentaol) is also an advantageous active ingredient for use in the present invention.

Plant extracts containing catechols may furthermore be advantageous, in particular extracts of green tea, such as, for example, extracts of leaves of the plants of the species Camellia spec., very particularly the tea strains Camellia sinensis, C. assamica, C. taliensis or C. irrawadiensis and crossings of these with, for example, Camellia japonica.

Further examples of preferred active ingredients include polyphenols and catechols from the group of (−)-catechol, (+)-catechol, (−)-catechol gallate, (−)-gallocatechol gallate, (+)-epicatechol, (−)-epicatechol, (−)-epicatechol gallate, (−)-epigallocatechol, and (−)-epigallocatechol gallate.

Flavone and its derivatives (often also collectively called “flavones”) may also be advantageous active ingredients for use in the present invention. They are characterized by the following basic structure (substitution positions indicated):

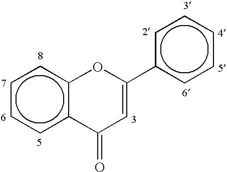

Some of the more important flavones, which may also preferably be employed in preparations according to the invention, are listed in Table 2 below:

TABLE 2

| OH substitution positions | | | | | | | |
|---|---|---|---|---|---|---|---|
| 3 | 5 | 7 | 8 | 2′ | 3′ | 4′ | 5′ |

| | | | | | | | | |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones as a rule occur in glycosylated form.

According to the invention, the flavones may preferably be selected from substances of the generic structural formula

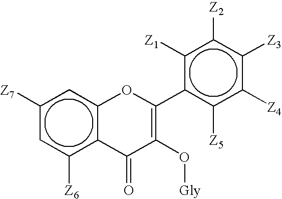

where Z₁ to Z₇ independently of one another are selected from H, OH, alkoxy and hydroxyalkoxy groups, where the alkoxy or hydroxyalkoxy groups can be branched or unbranched and can have about 1 to 18 C atoms, and where Gly is selected from mono- and oligoglycoside radicals.

The flavonoids may also advantageously be selected from substances of the structural formula

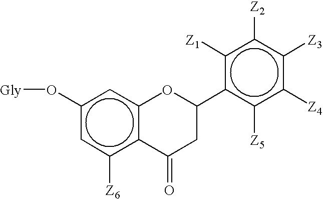

where Z₁ to Z₆ independently of one another are selected from H, OH, alkoxy and hydroxyalkoxy groups, where the alkoxy or hydroxyalkoxy groups can be branched or unbranched and can have about 1 to 18 C atoms, and where Gly is selected from the group mono- and oligoglycoside radicals.

Preferably, those structures may be selected from substances of the formula

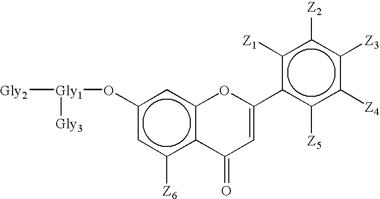

where Gly₁, Gly₂ and Gly₃ independently of one another are monoglycoside radicals. Gly₂ and Gly₃ can also individually or together be saturations by hydrogen atoms. Preferably, Gly₁, Gly₂ and Gly₃ independently of one another are selected from hexosyl radicals, in particular the rhamnosyl radicals and glucosyl radicals. However, other hexosyl radicals, for example allosyl, altrosyl, galactosyl, gulosyl, idosyl, mannosyl and talosyl may also be used advantageously. It may also be advantageous to use pentosyl radicals.

Advantageously, Z₁ to Z₅ independently of one another may be selected from H, OH, methoxy, ethoxy and 2-hydroxyethoxy groups, and the flavone glycosides may have the structure

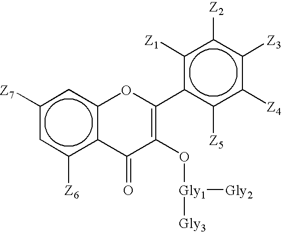

Particularly advantageously, the flavone glycosides for use in the invention may be selected from the group which is represented by the following formula:

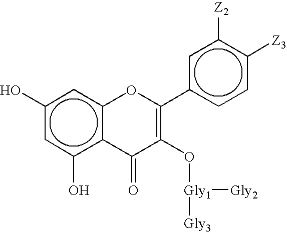

where Gly₁, Gly₂ and Gly₃ independently of one another are monoglycoside radicals. Gly₂ and Gly₃ can also individually or together be saturations by hydrogen atoms.

Preferably, Gly₁, Gly₂ and Gly₃ independently of one another are selected from hexosyl radicals, in particular the rhamnosyl radicals and glucosyl radicals. However, other hexosyl radicals, for example allosyl, altrosyl, galactosyl, gulosyl, idosyl, mannosyl and talosyl may also be used advantageously. It may also be advantageous a to use pentosyl radicals.

It may be particularly advantageous to select the flavone glycoside(s) from α-glucosylrutin, α-glucosyl-myricetin, α-glucosylisoquercitrin, α-glucosylisoquercetin and α-glucosylquercitrin. α-Glucosylrutin is particularly preferred.

Any one of naringin (aurantiin, naringenin 7-rhamnoglucoside), hesperidin (3′,5,7-trihydroxy-4′-methoxyflavanone 7-rutinoside, hesperidoside, hesperetin 7-O-rutinoside), rutin (3,3′,4′,5,7-pentahydroxyflavone 3-rutinoside, quercetin 3-rutinoside, sophorin, birutan, rutabion, taurutin, phytomelin, melin), troxerutin (3,5-dihydroxy-3′,4′,7-tris(2-hydroxyethoxy)flavone 3-(6-O-(6-deoxy-α-L-mannopyranosyl)-β-D-glucopyranoside)), monoxerutin (3,3′,4′,5-tetrahydroxy-7-(2-hydroxyethoxy)flavone 3-(6-O-(6-deoxy-α-L-mannopyranosyl)-β-D-glucopyranoside)), dihydrorobinetin (3,3′,4′,5′,7-penta-hydroxyflavanone), taxifolin (3,3′,4′,5,7-penta-hydroxyflavanone), eriodictyol 7-glucoside (3′,4′,5,7-tetrahydroxyflavanone 7-glucoside), flavanomarein (3′,4′,7,8-tetrahydroxyflavanone 7-glucoside) and isoquercetin (3,3′,4′,5,7-pentahydroxyflavanone 3-(β-D-glucopyranoside) may also be advantageous for use in the present invention.

It may also be advantageous to select the active ingredient(s) from ubiquinones and plastoquinones.

Ubiquinones may be represented by the structural formula

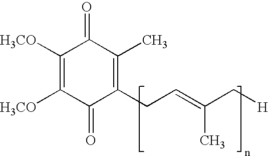

and are the most widespread and thus, the best investigated bioquinones. Depending on the number of the isoprene units linked in the side chain, ubiquinones are designated as Q-1, Q-2, Q-3 etc. or on the number of C atoms as U-5, U-10, U-15 etc. They preferably occur with certain chain lengths, for example in some microorganisms and yeasts with n=6. In most mammals including man Q10 predominates.

Particularly advantageous is coenzyme Q10, which may be represented by the following structural formula:

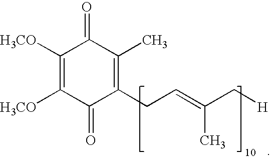

Plastoquinones have the structural formula

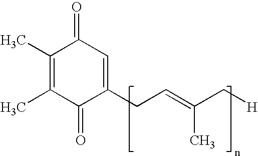

Plastoquinones differ in the number n of the isoprene radicals and are designated accordingly, for example PQ-9 (n=9). Furthermore, other plastoquinones having different substituents on the quinone ring exist.

Creatine and/or creatine derivatives are further preferred active ingredients for use in the present invention. Creatine has the following structure:

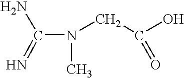

Preferred derivatives include creatine phosphate and creatine sulfate, creatine acetate, creatine ascorbate and the derivatives esterified on the carboxyl group by mono- or polyfunctional alcohols.

A further example of an advantageous active ingredient for use in the present invention is L-carnitine [3-hydroxy-4-(trimethylammonio)butyric acid betaine]. Acylcarnitines, which may be selected from substances of the following general formula

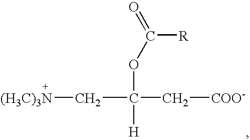

where R represents branched and unbranched alkyl radicals having up to about 10 carbon atoms are also advantageous active ingredients for use in the present invention. Propionylcarnitine and in particular, acetylcarnitine are preferred. Both enantiomers (D- and L-form) can be used advantageously in the present invention. It may also be advantageous to use any desired mixtures of enantiomers, for example a racemate of the D- and L-form.

Further advantageous active ingredients include sericoside, pyridoxol, vitamin K, biotin and aromatic substances.

Moreover, the active ingredients (one or more compounds) may also very advantageously be selected from hydrophilic active ingredients, in particular from the following group: alpha hydroxy acids such as lactic acid and salicylic acid and their salts such as, for example, Na lactate, Ca lactate, TEA lactate, urea, allantoin, serine, sorbitol, glycerol, milk proteins, panthenol, and chitosan.

The above list of active ingredients and active ingredient combinations, which may be used in the preparations according to the invention, is, of course, non-limiting and not exhaustive. The active ingredients can be used individually or in any desired combinations with one another.

The amount of such active ingredients (one or more compounds) in the preparations according to the invention may preferably be from about 0.001% to about 30% by weight, particularly preferably from about 0.05% to about 20% by weight, in particular from about 1 to about 10% by weight, based on the total weight of the preparation.

The emulsions according to the present invention may advantageously contain one or more preservatives. Advantageous preservatives for use in the present invention include, for example, formaldehyde-generating agents (such as, for example, DMDM hydantoin, which is obtainable, for example, under the trade name Glydant™ from Lonza), iodopropylbutyl carbamates (e.g., those obtainable under the trade names Glycasil-L, Glycasil-S from Lonza and/or Dekaben LMB from Jan Dekker), parabens (i.e., alkyl p-hydroxybenzoates, such as, e.g., methyl-, ethyl-, propyl- and/or butyl-paraben), phenoxyethanol, ethanol, benzoic acid and the like. Customarily, the preservation system, according to the invention, further advantageously may also include preservation aids, such as, for example, octoxyglycerol, Glycine soya, etc. The following table lists non-limiting examples of advantageous preservatives for use in the present invention:

TABLE 3

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na salt |
| E 218 | Methyl p-hydroxybenzoate |
| E 219 | Methyl p-hydroxybenzoate Na salt |
| E 220 | Sulphur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite) |
| E 230 | Biphenyl (diphenyl) |
| E 231 | Orthophenylphenol |
| E 232 | Sodium orthophenylphenolate |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Preservatives or preservation aids customary in cosmetics are furthermore advantageous, such as dibromodicyanobutane (2-bromo-2-bromomethyl-glutaronitrile), phenoxyethanol, 3-iodo-2-propynylbutyl carbamate, 2-bromo-2-nitro-propane-1,3-diol, imidazolidinylurea, 5-chloro-2-methyl-4-isothiazolin-3-one, 2-chloro-acetamide, benzalkonium chloride, benzyl alcohol, salicylic acid and salicylates.

It is in this case particularly preferred according to the invention if, as preservatives, iodopropylbutyl carbamates, parabens (methyl-, ethyl-, propyl- and/or butylparaben) and/or phenoxyethanol are employed.

One or more preservatives in a concentration of, for example, about 2% by weight or less than about 2% by weight, preferably about 1.5% by weight or less than about 1.5% by weight and particularly preferably about 1% by weight or less than about 1% by weight may advantageously be used in the emulsions according to the invention, in each case based on the total weight of the preparation.

The emulsions according to the invention may advantageously contain one or more conditioners. Preferred conditioners include, for example, all compounds which are listed in the International Cosmetic Ingredient Dictionary and Handbook (Vol 4, editors: R. C. Pepe, J. A. Wenninger, G. N. McEwen, The Cosmetic, Toiletry, and Fragrance Association, 9th edition, 2002) under section 4 under the keywords Hair-Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all compounds listed in EP 0934956 (pp. 11-13) under water soluble conditioning agent and oil soluble conditioning agent. Some of these compounds are listed under the constituents of the aqueous phase and of the oil phase especially. Further advantageous conditioners according to the invention include, for example, the compounds mentioned according to the international nomenclature for cosmetic ingredients (INCI) as polyquaternium (in particular polyquaternium-1 to polyquaternium-56).

All concentrations indicated herein relate to the total weight of the cleansing emulsion, if not stated otherwise.

According to the invention, the emulsions may advantageously contain glitter and/or other effect substances (e.g. color streaks).

The emulsions according to the invention may advantageously be stored in a bottle or squeeze bottle and applied therefrom. The emulsions according to the invention may also advantageously be stored in tubes and applied therefrom. Storage in a 2-chamber pack is another non-limiting example of an advantageous storage form of the emulsions of the present invention.

The emulsions according to the present invention may also advantageously be applied to a preferably insoluble substrate as an impregnation. Suitable substrates include smooth and surface-structured substrates. Surface-structured substrates are preferred. Insoluble substrates in the form of tissues are preferably employed, which comprise nonwoven material, in particular water jet-consolidated and/or water jet-imprinted nonwoven material. The substrates may advantageously also be constructed as a pad, perforated nonwoven material or net.

According to the invention, the emulsion may advantageously be present in the form of a cream (also called a mousse) foamed by means of a gas, for example in a cosmetic jar.

According to the invention, the emulsion may also advantageously be present in a compressed gas bottle or an aerosol container. According to the invention, pressurized gas bottles are preferred which contain the preparation in the interior of a flexible sachet, whose contents are pressed outward by the overpressure which acts on the sachet in the pressurized gas bottle. Particularly preferably, the preparation in such a case contains a further readily volatile component such as pentane, whereby “afterfoaming” preparations can be produced.

Also advantageous according to the invention is the storage and application of the preparation using a pump spray apparatus or using a pump dispenser (“pump foamer”).

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice. All quantitative data, proportions and percentages are, if not stated otherwise, based on the weight and of the total amount or on the total weight of the preparations.

| | Example No. | | | | |
|---|---|---|---|---|---|
| | 1 | 2 | 3 | 4 | 5 |
| Sodium laureth sulfate | 7.00 | 10.00 | — | 9 | — |
| Cocamidopropylbetaine | 3.00 | — | — | 2 | 3 |
| Sodium myreth sulfate | — | — | 5 | — | 6 |
| Alkyl polyglucosides | 2.00 | — | — | — | 2.5 |
| Polyacrylate | 0.50 | 0.60 | 0.7 | 0.35 | 0.45 |
| PEG-7 glyceryl cocoate | 1.00 | — | 1.00 | — | — |
| Quaternary ammonium salt of | — | — | — | 0.10 | — |
| hydroxyethyl-cellulose | | | | | |
| Talc | 0.50 | — | — | — | — |
| Paraffin oil | 25 | 35 | 40 | 30 | 45 |
| Soybean oil | 18 | 6 | 5 | 13 | — |
| Almond oil | — | 1 | — | — | — |
| Jojoba oil | — | 1 | — | — | 0.5 |
| Unispheres (lactose + cellulose + | — | 0.2 | — | — | — |
| hydroxypropylmethylcellulose + CI 77007) | | | | | |
| Phenoxyethanol + methylparaben + | 1.00 | 0.8 | 1.00 | 1.00 | 1.00 |
| butylparaben + ethylparaben + | | | | | |
| isobutyl-paraben + propylparaben | | | | | |
| Butylhydroxytoluene | 0.05 | 0.05 | 0.05 | 0.05 | — |
| NaOH | q.s. | q.s. | q.s. | q.s. | q.s. |
| Perfume | 1.5 | 1.2 | 1 | 1 | 1.2 |
| Water | to 100 | to 100 | to 100 | to 100 | to 100 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cosmetic or dermatological cleansing emulsion comprising:
(a) from about 2% to about 17% by weight of at least one of sodium laureth sulfate and sodium myreth sulfate;
(b) from about 0.20% to about 0.74% by weight of one or more polyacrylates selected from anionic homopolymers and anionic copolymers of at least one of acrylic acid, an alkylated acrylic acid and esters thereof;
(c) from about 42% to about 51% by weight an oil phase comprising
(i) from about 25% to about 50% by weight of a paraffin oil,
(ii) from about 0.5% to about 25% by weight one or more oils having a polarity of from about 5 to about 50 mN/m;
the emulsion having a viscosity of from about 500 to about 3,500 mPa s at 100 s⁻¹.

2. The emulsion of claim 1, wherein the emulsion comprises from about 4% to about 15% by weight of (a).

3. The emulsion of claim 2, wherein the emulsion comprises at least about 5% by weight of (a).

4. The emulsion of claim 2, wherein the emulsion comprises not more than about 10% by weight of (a).

5. The emulsion of claim 2, wherein the emulsion comprises sodium laureth sulfate.

6. The emulsion of claim 1, wherein the emulsion comprises sodium myreth sulfate.

7. The emulsion of claim 1, wherein the emulsion comprises from about 0.30% to about 0.70% by weight of (b).

8. The emulsion of claim 1, wherein the emulsion comprises at least about 0.35% by weight of (b).

9. The emulsion of claim 1, wherein the emulsion comprises from about 43% to about 46% by weight of (c).

10. The emulsion of claim 1, wherein the emulsion comprises at least about 43% by weight of (c).

11. The emulsion of claim 10, wherein the emulsion comprises not more than about 45.5% by weight of (c).

12. The emulsion of claim 9, wherein the emulsion comprises from about 30% to about 45% by weight of (c)(i).

13. The emulsion of claim 9, wherein the emulsion comprises from about 5% to about 20% by weight of (c)(ii).

14. The emulsion of claim 1, wherein the emulsion comprises at least about 1% by weight of (c)(ii).

15. The emulsion of claim 13, wherein (c)(ii) has a polarity of from about 10 to about 45 mN/m.

16. The emulsion of claim 1, wherein (c)(ii) comprises at least one fatty acid triglyceride.

17. The emulsion of claim 16, wherein the fatty acid triglyceride comprises at least one of soybean oil and almond oil.

18. The emulsion of claim 12, wherein (c)(ii) comprises at least one of a hydrocarbon, a fatty acid triglyceride, a silicone oil and a carboxylic acid ester.

19. The emulsion of claim 1, wherein the weight ratio (b): (c)(i) is from about 1:125 to about 1:68.

20. The emulsion of claim 12, wherein the weight ratio (b): (c)(i) is from about 1:100 to about 1:57.

21. The emulsion of claim 1, wherein the emulsion has a viscosity of from about 700 to about 3,000 mPa s at 100 s⁻¹.

22. The emulsion of claim 1, wherein the emulsion further comprises at least one surfactant.

23. The emulsion of claim 22, wherein the at least one surfactant comprises a surfactant having an HLB value of higher than about 25.

24. The emulsion of claim 22, wherein the at least one surfactant comprises a surfactant having an HLB value of higher than about 35.

25. The emulsion of claim 22, wherein the emulsion comprises from about 1% to about 30% by weight of the at least one surfactant.

26. The emulsion of claim 1, wherein the emulsion further comprises at least one active ingredient.

27. The emulsion of claim 26, wherein the at least one active ingredient is present in a concentration of from about 0.001% to about 10% by weight.

28. A cosmetic or dermatological cleansing emulsion comprising:
(a) from about 5% to about 10% by weight of at least one of sodium laureth sulfate and sodium myreth sulfate;
(b) from about 0.30% to about 0.70% by weight of one or more polyacrylates selected from anionic homopolymers and anionic copolymers of at least one of acrylic acid, an alkylated acrylic acid and esters thereof;
(c) from about 43% to about 46% by weight an oil phase comprising
(i) from about 30% to about 45% by weight of a paraffin oil,
(ii) from about 5% to about 20% by weight of one or more oils having a polarity of from about 10 to about 45 mN/m;
the emulsion having a viscosity of from about 700 to about 3,000 mPa s at 100 s⁻¹.

29. The emulsion of claim 28, wherein the emulsion comprises sodium laureth sulfate.

30. The emulsion of claim 28, wherein the emulsion comprises sodium myreth sulfate.

31. The emulsion of claim 28, wherein the emulsion comprises at least about 0.35% by weight of (b).

32. The emulsion of claim 31, wherein the emulsion comprises not more than about 45.5% by weight of (c).

33. The emulsion of claim 31, wherein (c)(ii) comprises at least one of soybean oil and almond oil.

34. The emulsion of claim 32, wherein the weight ratio (b): (c)(i) is from about 1:100 to about 1:57.

35. The emulsion of claim 28, wherein the emulsion further comprises from about 10% to about 20% by weight of at least one surfactant having an HLB value of higher than about 25.

36. The emulsion of claim 35, wherein the emulsion further comprises from about 0.05% to about 10% by weight of at least one active ingredient.

37. A foamed mousse which comprises the emulsion of claim 1.

38. A compressed gas bottle which comprises the emulsion of claim 1.

39. An aerosol container which comprises the emulsion of claim 1.

40. A foam bath which comprises the emulsion of claim 1.

41. A shower bath which comprises the emulsion of claim 1.

42. A tub bath which comprises the emulsion of claim 1.

43. A face cleanser which comprises the emulsion of claim 1.

44. A hair shampoo which comprises the emulsion of claim 1.

45. A method of cleansing the skin and its appendages, wherein the method comprises the application of a product which comprises the emulsion of claim 1 onto at least parts of the skin.

46. A process for making a cosmetic or dermatological cleansing emulsion, which process comprises combining
(a) from about 2% to about 17% by weight of at least one of sodium laureth sulfate and sodium myreth sulfate;
(b) from about 0.20% to about 0.74% by weight of one or more polyacrylates selected from anionic homopolymers and anionic copolymers of at least one of acrylic acid, an alkylated acrylic acid and esters thereof;
(c) from about 42% to about 51% by weight an oil phase comprising
(i) from about 25% to about 50% by weight of a paraffin oil,
(ii) from about 0.5% to about 25% by weight one or more oils having a polarity of from about 5 to about 50 mN/m;
to form an emulsion having a viscosity of from about 500 to about 3,500 mPa s at 100 s⁻¹.
